(19) United States
(12) Patent Application Publication
(10) Pub. No.: US 2018/0185294 A1
(43) Pub. Date: Jul. 5, 2018

(54) MYCO Capsule

(71) Applicant: Connor Francis Leach, Roswell, NM (US)

(72) Inventor: Connor Francis Leach, Roswell, NM (US)

(21) Appl. No.: 15/438,898

(22) Filed: Feb. 22, 2017

Related U.S. Application Data
(60) Provisional application No. 62/442,458, filed on Jan. 5, 2017.

(51) Int. Cl. A61K 9/48

(52) U.S. Cl. CPC A61K 9/4825

(57) ABSTRACT

MYCO Capsules are the next wave of innovation in the pharmaceutical industry, by improving individual company spending budget costs, and increasing medication production rates across the world, through giving the user a new digestive medicine method, which will best fit the users diagnosis, anatomy, philosophy, and medicine spending budget. Where reduction costs of medicine aspire, lower health insurance plans will arise. As the cost of health insurance plans decrease, more users will be able to afford health insurance with plans that cover the use of MYCO Capsules.

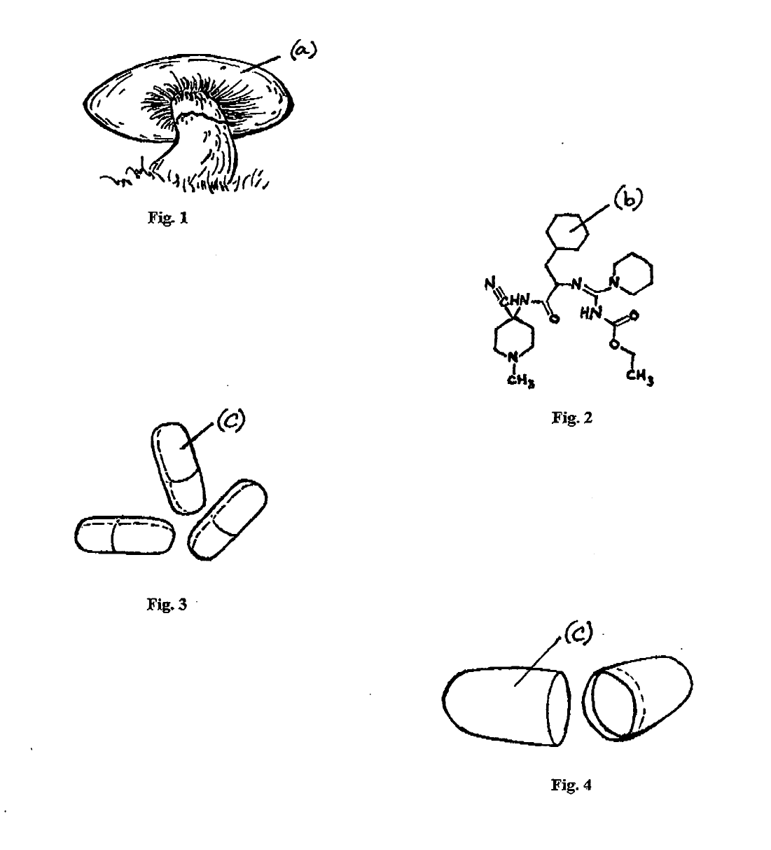

### FIELD OF INVENTION

The present invention regards to the pharmaceutical industry.

### ISSUES

There are two types of capsules on the market, gelatin and hypromellose, (animal and plant), that both have problematic issues. Gelatin capsules are derived from a genetically modified production of animal connective tissues. Gelatin contains animal skin and crushed bones. Gelatin capsules breakdown and dissolve in humidity. Gelatin is formed from boiled animal parts such as cattle, pigs, and fish that are hazardously transferred, or slaughtered. Gelatin capsules have a low melting point between 77 degrees to 104 degrees Fahrenheit. Hypromellose is only made from pollen and pine. 18% of humans are allergic to pollen and 2% of humans have allergens to pine, summing an average 19% of the human population that are allergic to the vegetarian capsule alternative. Hypromellose is formulated from cellulose, which does not digest in the human body. Cellulose is not an essential nutrient and rushingly pushes food through the digestive system, processing fermentation, causing gas in the large intestine from intestinal bacteria. Hypromellose dissolves at 500 degrees Fahrenheit, forever making its formula humanly impossible to digest. Cellulose is very expensive, which is why hypromellose is genetically modified in its manufacturing, perceiving the vegetarian alternative to be a greater health risk than hazardous gelatin. A hypromellose capsule is three times the price of a gelatin capsule. The common cause of allergic reactions to vaccinations are from gelatin allergens.

### BACKGROUND

The present invention, MYCO Capsule, seeks to provide a solution to the issues of gelatin and hypromellose capsules, through an alternative mycoprotein capsule.

### BRIEF SUMMARY

Mycoprotein is known as fungal protein, first produced in the UK during 1980, eleven years after Fungi was named as its own Kingdom. Myco is the greek word for Fungus. Fungi-Culture is the process of producing fungi in medicine. Myco is made of chitin, glucans, and protein. (a) Myco relates to humans more than plants, by storing food as glycogen, like in animals. (b) Mycoprotein is a strain of protein derived from fungi, when grown properly, mycoprotein can double its mass every 5 hours. (c) MYCO Capsules are made from mycoprotein.

### BRIEF DESCRIPTION OF THE DRAWINGS

See drawings on “only black and white drawings page.”

### DETAILED DESCRIPTION

MYCO Capsules are efficiently produced. Mycoprotein is a comparable substance to gelatin with a similar component structured formula made up of carbon dioxide, hydrogen, nitrogen, and oxygen. Mycoprotein is freed from fat with ether. The putrefactive bacterial cells obtained by decantation liquid cultures are dissolved into 50 parts of potash solution at 0.5%, then heated for several hours above 100 degrees celsius before being filtered. The filtrate is acidified with dilute hydrochloric acid and precipitated by some added rock salt. After being washed with saturated salt solution, and dried below 100 degrees celsius, the substance is then freed from self through being washed with water. Mycoprotein is heated above high temperatures, shaped, and cooled into MYCO Capsules. (b) Formula: C25H42N6O3

### BASIC DESCRIPTION OF HOW IT IS MADE

Mycoprotein is made in fermenters like as seen in a brewery through adding oxygen, nitrogen, glucose, and minerals to fungi. Fusarium venenatum is the classification of fungus species where strains of mycoprotein are formulated. The mycoprotein, derived from Fusarium venenatum, is then heated above melting temperatures of 100 degrees, and cooled into a solid capsule structure, creating MYCO Capsules.

### HOW IT WORKS

MYCO Capsules are the better alternative product that markets to reduce pharmaceutical spending of capsule production through cheaper energy and cleaner energy. MYCO Capsules will reduce the cost of the capsule market by half with providing the energy security needed for pharmaceutical companies, from less expenses, and will provide safe laboratories without hazardous materials such rotting dead animal bodies to make gelatin, and implement reductions of water waste used from creating genetically modified hypromellose. The production cycle of MYCO Capsules will utilize less space, time, and energy. This entails producing medicine to the masses of the world quicker, with a speedy growing mycoprotein process, where as the world will have more medicine as production rates sky rocket. In result, epidemic percentages will decrease with having more medicine made available to the public.

### COMPARABLE CHEMISTRY

Mycoprotein compares a similar chemical formula to gelatin and hypromellose. (Gelatin formula: C612O6, Hypromellose formula: C56H108O30)

### MOST IMPORTANT PREFERRED ASPECTS

MYCO Capsules have major health benefits for users. Mycoprotein has diary fibre, unlike in gelatin, as well as less amount of lipids. Mycoprotein has a lower protein mass than gelatin, which holds a higher quality of protein than in beef, measured at 0.99 in mycoprotein and measured at 0.92 in beef. MYCO Capsules will dissolve faster in the digestive system than gelatin, unlike hypromellose which does not digest in humans whatsoever. Mycoprotein is naturally low in fats and saturates with zero cholesterol, zero calories, and zero mycotoxins. Mycoprotein is a Non-GMO product, unlike gelatin and hypromellose, therefor mycoprotein is not genetically modified and is fundamentally vegan.

### EMBODIMENT

The inventor of the MYCO Capsule excludes any making, using, distributing, or selling of the invention without licensing the right to use the patent in return of a contracted profit. Any and all fungi and or fungus capsules attempted to me made and sold will be a violation against the patent. All rights are reserved to the rightful inventor of this patent for the invention of all fungus capsules, fungi capsules, mycoprotein capsules, and MYCO Capsules.

1. MYCO Capsules are the healthiest pharmaceutical capsule choice for all diabetics.

2. MYCO Capsules prove health benefits in digestion.

3. MYCO Capsules have low sodium.

4. MYCO Capsules have low fats.

5. MYCO Capsules have zero calories.

6. MYCO Capsules have zero mycotoxins.

7. MYCO Capsules have zero cholesterol levels.
